SEQUENCE LISTING (5 sequences)

SEQ ID NO: 1 - protein, 643 aa, Trypanosoma cruzi
MLAPGSSRVELFKRKNSTVPFEDKAGKVTERVVHSFRLPALVNVDGVMVAIADARYDTSNDNSLIDTVAKYSVDDGETWETQIAIKNSRVSSVSRVVDPTVIVKGNKLYVLVGSYYSSRSYWSSHGDARDWDILLAVGEVTKSIAGGKITASIKWGSPVSLKKFFPAEMEGMHTNQFLGGAGVAIVASNGNLVYPVQVTNKRKQVFSKIFYSEDDGKTWKFGKGRSDFGCSEPVALEWEGKLIINTRVDWKRRLVYESSDMGNTWVEAVGTLSRVWGPSPKSDHPGSQSSFTAVTIEGMRVMLFTHPLNFKGRWLRDRLNLWLTDNQRIYNVGQVSIGDENSAYSSVLYKDDKLYCLHEINTDEVYSLVFARLVGELRIIKSVLRSWKNWDSHLSSICTPADPAASSSESGCGPAVTTVGLVGFLSGNASQNVWEDAYRCVNASTANAERVRNGLKFAGVGGGALWPVSQQGQNQRYRFANHAFTLVASVTIHEAPRAASPLLGASLDSSGGKKLLGLSYDEKHQWQPIYGSTPVTPTGSWETGKRYHVVLTVANKIGSVYIDGELLEGSGQTVVPDGRTPDISHFYVGGYGRSDMPTISHVTVNNVLLYNRQLNTEEIRTLFLSQDLIGTEAHMDSSSDTSA

SEQ ID NO: 2 - DNA, 20 bp, Artificial sequence
  PCR Primer
gctcttgcccacamgggtgc

SEQ ID NO: 3 - DNA, 21 bp, Artificial sequence
  PCR Primer
ccaagcagcggatagttcagg

SEQ ID NO: 4 - DNA, 25 bp, Artificial sequence
  PCR Primer
tccctctcatcagttctatggccca

SEQ ID NO: 5 - DNA, 27 bp, Artificial sequence
  PCR Primer
cagcaagcatctatgcacttagacccc

(19) United States
(12) Patent Application Publication
(10) Pub. No.: US 2019/0351035 A1
(43) Pub. Date: Nov. 21, 2019

(54) VACCINE AGAINST TRYPANOSOMA CRUZI INFECTION

(71) Applicant: Carlos Baeremaecker, Montevideo (UY)

(72) Inventor: Carlos Baeremaecker, Montevideo (UY)

(21) Appl. No.: 16/311,597

(22) PCT Filed: May 2, 2017
(86) PCT No.: PCT/CL2017/050020

§ 371 Date: Dec. 19, 2018

(30) Foreign Application Priority Data
May 4, 2016 (AR) P20160101264

(51) Int. Cl. A61K 39/005; C07K 14/44; C12N 9/24

(52) U.S. Cl. CPC A61K 39/005; C12N 9/24; C07K 14/44

(57) ABSTRACT

A vaccine composition against infection by Trypanosoma cruzi comprising at least one Trypanosoma cruzi trans-sialidase mutant protein (SEQ 1) and, as adjuvant, a mixture of a highly purified mineral oil and mannide monooleate.

### SEQUENCE LISTING

The sequence listing submitted via EFS, in compliance with 37 CFR §1.52(e)(5), is incorporated herein by reference. The sequence listing text file submitted via EFS contains the file “VERA-0003_ST25.txt”, created on Dec. 19, 2018, which is 6,027 bytes in size.

### FIELD OF THE INVENTION

The present invention relates to a vaccine against Trypanosoma cruzi infection (T. cruzi), useful in the prevention and/or treatment of Chagas disease.

In particular, the present invention relates to a vaccine composition comprising at least one T. cruzi trans-sialidase mutant protein and, as adjuvant, a mixture of highly purified mineral oil and mannide monooleate. In a preferred embodiment of the invention, said highly purified mineral oil is marketed as Drakeol® 6VR. In another preferred embodiment of the present invention, the adjuvant is commercially available as Montanide® ISA 51 VG (Seppic, France).

In an especially preferred embodiment of the invention, the vaccine comprises a Trypanosoma trans-sialidase mutant protein having the sequence identified as SEQID NO:1 and, as adjuvant, Montanide® ISA 51 VG.

The vaccine composition according to the invention can be used against parasitemia and at the same time, to protect tissue from damage caused by parasites.

### BACKGROUND OF THE INVENTION

Chagas disease, also known as American trypanosomiasis or “Chagas disease”, is a parasitic disease transmitted by Trypanosoma cruzi, a parasite related African trypanosomes. The most common way of contracting the disease through contact with the feces of triatomines (Triatoma infestans (in our region), also known by the names of assassin bug, bedbug besucona, benchuca, chipo o barbeiro), which feeds on blood of humans and animals. Once the parasite reaches the wound created by the insect, it spreads through the body invading host cells.

This disease is one of the major health problems in Latin America, where approximately 8 to 10 million people could be infected. Risk factors for Chagas disease include, among others, living in Mexico, Central America or South, poverty, inhabiting shanties where the bloodsucking insects can stay in the walls and blood transfusion from a person who has the parasite, even if the donor does not have active disease. There is also vertical transmission (mother/child) and contaminated food.

Chagas disease has two phases: acute and chronic. The first is usually presented with mild symptoms, children under 2 years meningitis may develop heart disease or meningitis (1% of cases). Inflammation may appear at the site of entry of the parasite and the infection site may show redness. If infection occurs through the conjunctiva the sign Romagna (pathognomonic) is generated.

As the parasite spreads from the site of entry, the patient has fever, malaise, and generalized swelling of the lymph nodes. Also, the liver and spleen may become enlarged. The disease decreases its intensity after its acute phase and becomes chronic without further symptoms for many years. In 30% of cases the symptoms manifest belatedly, they appear as heart disease (cadiomiopatia) and digestive (megaviscera).

Patients may have congestive heart failure, while the first symptom of digestive disorder can be difficulty in swallowing, which can lead to malnutrition. Patients experiencing parasitic infection of the colon may experience abdominal pain and constipation. Heart disease is usually the cause of death. Approximately 70% of patients with Chagas' disease die from heart failure due to severe heart damage.

Within the host cell, the parasite becomes amastigote that can multiply very rapidly changing back into infectious trypomastigotes. Shortly thereafter, the host cells burst, freeing the parasites that will be able to infect other cells. The molecular mechanism by which the parasite infects host cells is very complex and has been studied over the years. It has been shown that T. cruzi expresses a unique enzyme in its kind that transfers sialic acid, which is capable of hydrolyzing sialic acids with α-2.3 joints and transfer them to terminal β-galactose residues: trans-ialidase enzyme (TS). A portion of this enzyme was initially identified as an important antigen (SAPA, Shed Acute Phase Antigen) in the acute phase of Chagas disease (CITE Pollevick 1991) and then characterize the novel enzymatic activity of the complete protein (Pollevick G D, Affranchino. J L, Frasch A C, D O Sanchez). The complete sequence of a shed acute-phase antigen of Trypanosoma cruzi Mol Biochem Parasitol 1991 August; 47 (2): 247-50 PubMed PMID: 1840626. And Parodi A J, Pollevick G D, Mautner M, Buschiazzo A, Sanchez D O, Frasch A C Identification of the gene(s) coding for the trans-sialidase of Trypanosoma cruzi EMBO J. 1992 May; 11 (5): 1705-1710 PubMed PMID: 1,374,711; PubMed Central PMCID: PMC556627).

This enzyme can be anchored in the cell membrane of the parasite through a GPI anchor or, after cleavage by a lipase enzyme of the parasite, may remain in the blood. TS plays an important role in the infection cycle of T. cruzi because it allows the invasion of host cells. It has been shown that when the TS activity is inhibited (for example using lines of mutant cells lacking sialic acid on its surface (Ciavaglia M., de Carvalho and Souza T U W. (1993) “Interaction of Trypanosoma cruzi cells With altered glycosylation patterns “, Biochem Biophys res Commun 193, 718-721; Ming M. et al (1993)” Mediation of Trypanosoma invasion by sialic acid on the host cell and trans-siaiidase on the trypanosorne “Mol. Biochem. Farasital. 59, 243-252 and RPS Schenkman et al. (1993) “Mammalian cell sialic acid Enhances Trypanosoma cruzi invasion”, Infect. Immun. 61, 898-902) or blocking acceptor molecules on the surface of the parasite (Yoshida N. et al., (1989) “Metacyclic neutralizing effect of 10D8 monoclonal antibody directed to the 35- and 50-kilodalton Surface glycoconjugates of Trypanosoma cruzi”, Mol. Biochem. Parasitol. 39, 39-46 and R. Ruiz et al. (1993) “The 35/50 kDa surface antigen of Trypanosoma cruzi metacyclic trypomastigotes, an adhesion molecule in host cell invasion Involved” Parasitol. Immunol. 15, 121- 125) it is possible to inhibit the invasion of host cells by the parasite. In addition, the TS plays a role in the defense mechanism of the parasite against host immune system, as it is used to cover the surface of the parasite with sialic acid molecules, making it difficult for the immune system to detect the parasite.

As the TS enzyme plays such an important cycle of infection and defense function, the parasite developed various methods to protect the enzyme against the host's immune system. First, the parasite expresses more than 200 different TS of which only about 15 are active (EI-Sayed N M et al., 2005, “The genome sequence of Trypanosoma cruzi, etiologic agent of Chagas disease,” Science 309 (5733), 409- 415). This fact makes it difficult for the immune system to inhibit invasion of host cells by the parasite in the normal cycle of infection, since parasites and their TS are maintained only for a relatively short time in the bloodstream, then they enter the host cell, where they are protected from the immune system. In addition, TS have a very long immunodominant extension of SAPA repetitions, which drive antibodies away from the catalytic site of the enzyme.

The drugs traditionally used to treat Chagas disease are Nifurtimox and Benznidazole. These drugs work only in early chronic and acute phase of the disease, but not in the chronic phase. Between the two drugs previously mentioned, Benznidazole is usually the one preferred to treat Chagas disease as it has been shown to have better efficacy and better tolerance than Nifurtimox. However, due to their limited efficiency and its many side effects, these drugs have a limited use.

Thus, it is proposed that vaccination could provide a solution to these problems. Vaccination could be much more effective than existing drugs to treat chronic patients and also could have an effect in preventing the onset and progression of the disease.

Due of its central function in the infection and to the fact that the enzyme is not only exposed on the cell surface but also is present as a free molecule in the blood, it proves to be a good target for antibody. Thus, the TS may be a good antigen candidate for the production of a vaccine against Chagas disease. In this respect, considering that there are no TS counterparts in humans, the antibodies generated by the patient's immune system would be specific of the parasite, so the vaccine should not have relevant side effects.

In the patent application WO 2007/107488 PCT A2 has been reported that mutants with limited enzymatic activity could be used as vaccines. However, these mutants did not exhibit immunogenic activity and adequate efficacy so as to be formulated into a suitable vaccine composition against infection by Trypanosoma cruzi to be administered in humans and animals.

### BRIEF DESCRIPTION OF THE INVENTION

The present invention relates to a vaccine against infection by Trypanosoma cruzi (T. cruzi), useful in the prevention and/or treatment of Chagas disease.

In particular, the present invention relates to a vaccine composition comprising at least one T. cruzi trans-sialidase (TS mut) mutant protein and a mixture of highly purified mineral oil and mannide monooleate as adjuvant.

### BRIEF DESCRIPTION OF THE FIGURES

FIG. 1. Results obtained in terms of survival. Three groups of 12, 60 days of age male BALB/ci mice were used. The animals received three doses of 15 ug of TS (Group A: TS wild (WT), Group B: TS mut (SEQID NO:1) and group C: control). The first dose was emulsified in CFA (1:1 vol:vol) and the other two in IFA (1:1, vol:vol) and administered via subcutaneous (s.c) at two week intervals. Control means saline solution in adjuvant. The animals were infected with a virulent strain of T. cruzi DTU TCVI (RA), 500 blood trypomastigotes intraperitoneally (ip). Parasitaemia and mortality were monitored for 60 days was. A fourth group of animals (n=7) was immunized with TS obtained in E. coli (TScoli).

FIG. 2. Parasitemia values obtained in mice pertaining to the groups of FIG. 1. The parasitemia was determined by counting the parasites in a hemocytometer (Neubauer). Values are expressed as parasites/ml.

FIG. 3. Summary of results (number of equivalents of parasites/ng DNA) obtained by RT PCR in the tissues of the following study groups. GI: TS mut (SEQID NO:1); G2: TS WT; G3: TS coli; G: Control.

FIG. 4. Quantification of IL2, IL4 and IFNg by ELISA. (A, C, E) Each point represents quadruplicate values for each supernatant. (B, D, F) Each point represents the average of quadruplicate. Cultures were harvested 24 hours after adding 5 ug/ml of TSmut (SEQID NO:1). Control: Animals immunized with PBS/ISA51. TSmut: Animals immunized with TSmut/ISA51. By ex-vivo restimulation the specific response to the antigen was characterized as a clear Th1 response by significant production of IFNg from treated animals compared to control animals (FIGS. 4E and 4F).

FIG. 5. Evaluation of specific anti-TS antibodies, induced by immunization with TS mut. Groups of 12 male Balb/ci mice were immunized with TS mut/ISA51. Three s.c. doses were administered according to the scheme above and 15 days after the last dose specific IgG1 and IgG2a induction was tested through ELISA.

FIG. 6. Results obtained in terms of parasitemia of mice immunized with WT and TS mut using Freund's adjuvant or ISA 51. The animals were challenged 45 days after the first immunization with a T. cruzi DTU TcVI (RA) virulent strain, 500 ip blood trypomastigotes. Parasitemia and mortality were monitored for 60 days. Parasitemia was determined by counting the parasites in a hemocytometer (Neubauer). Values are expressed as parasites/ml.

### DETAILED DESCRIPTION OF THE INVENTION

The present inventors have found that, surprisingly, it is possible to obtain a vaccine against infection by Trypanosoma cruzi (T. Cruzi) having an immunogenic activity and an adequate efficacy for the treatment of humans and animals, comprising at least one trans-sialidase (TS mut) mutant protein of Trypanosoma cruzi and, as adjuvant, a mixture of a highly purified mineral oil and mannide monooleate.

In a preferred embodiment of the invention, said highly purified mineral oil is marketed as Drakeol® 6VR. In another preferred embodiment of the invention, the adjuvant is Montanide® ISA 51 VG (Seppic, France).

In an especially preferred embodiment of the invention, the vaccine composition comprises a trans-sialidase mutant protein of Trypanosoma cruzi of the sequence identified as SEQID NO:1 and, as adjuvant, Montanide® ISA 51 VG.

The vaccine composition according to the invention can be used against parasitemia and at the same time, to protect tissue from damage caused by parasites.

In particular, the vaccine composition according to the invention comprises, preferably a transsialidase mutant protein from Trypanosoma cruzi comprising SEQID NO:1.

### EXAMPLES

Test 1

All studies were conducted according to the standards set by CICUAE (Institutional Committee for the Care and Use of Experimental Animals), National University of San Martin (UNSAM). The animals were confined in a facility contained BSL3 at the Biotechnology Research Institute “Dr. Rodolfo A. Ugalde” (IIB), UNSAM, Buenos Aires, Argentina, where they were housed in individual cages, ventilated for two weeks before immunization. Each animal was labeled separately.

Three groups, each of them containing 12, 60 days of age male BALB/cJ mice were used. The animals received three doses of 15 ug of TS (Group A: TS wild (WT), Group B: TS mut and group C: control). The first dose was emulsified in CFA it (1:1 vol:vol) and the other two in IFA (1:1, vol:vol) and administered s.c. in two week intervals. Control means saline solution in adjuvant.

Animals were infected with a virulent strain of T. cruzi DTU TcVI (RA), 500 i.p. blood trypomastigotes. Parasitemia and mortality were monitored for 60 days. The parasitemia was determined by counting the parasites in a hemocytometer (Neubauer). Values are expressed in parasites/ml.

Group A
n = 12
Days

| 1 | 14 | 28 | 42 | 102 |
|---|---|---|---|---|
| WT/CFA | WT/IFA | WT/IFA | Challenge | Parasitemia/Mortality |

Group B
n = 11
Days

| 1 | 14 | 28 | 42 | 102 |
|---|---|---|---|---|
| TSmut/ | TSmut/ | TSmut/ | Challenge | Parasitemia/Mortality |
| CFA | IFA | IFA | | |

Group C
n = 12
Days

| 1 | 14 | 28 | 42 | 102 |
|---|---|---|---|---|
| Saline/CFA | Saline/ | Saline/ | Challenge | Parasitemia/Mortality |
| | IFA | IFA | | |

A fourth group of animals (n=7) was immunized with TS obtained in E. coli.

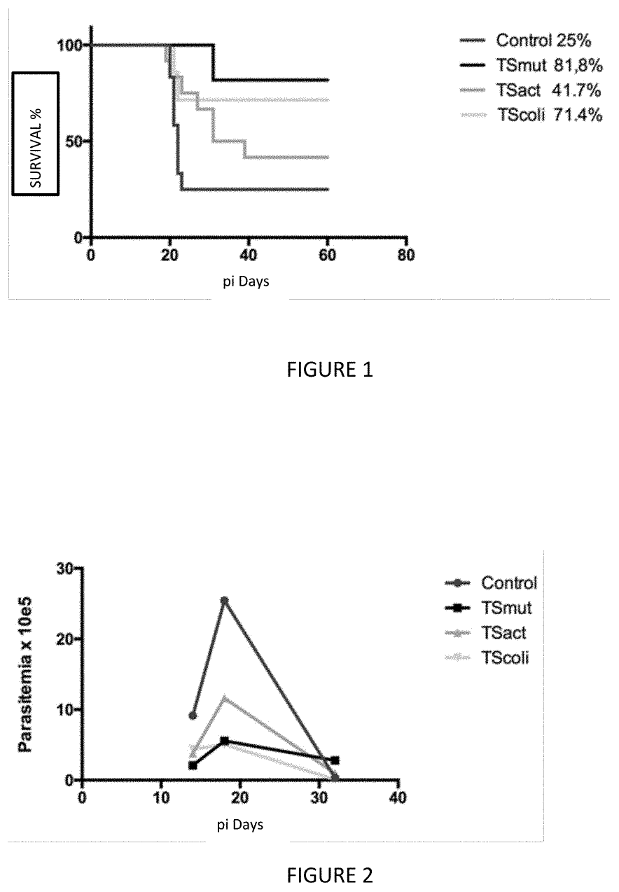

The results obtained in terms of survival and parasitemia are shown in FIGS. 1 and 2 respectively.

Extension of Test 1: TS mut Protective Capacity

Those mice that survived this test were challenged again 102 days after being infected. This time, the challenge was performed with the same strain of T. cruzi parasites but with 10,000 r i.p. blood trypomastigotes. All animals survived for 60 days after the second challenge, and at that time they were sacrificed. No parasitemia was observed in any of the animals when evaluated by the fresh drop test.

Histopathology Tests:

Firstly, the level of inflammation on cuts stained with hematoxylin/Eosin (HE) were evaluated. For this, skeletal muscle and heart of animals infected with T. cruzi were obtained and were fixed in 10% formaldehyde in PBS and embedded in paraffin. 5 um cuts were made, which were stained with HE. Then single-blind observations by light microscopy on coded preparations were made. The presence or absence of parasitized cells and inflammatory infiltrates in tissues was recorded Inflammation was qualitatively evaluated according to the presence or absence of necrosis of muscle cells and polymorphonuclear leukocyte in infiltrates (active or chronic inflammation, respectively) and semiquantitatively evaluated at low-power examination according to the distribution (focal confluent or diffuse and amount of inflammatory cells (1+for only one inflammatory focus, 2+non-confluent multiple inflammatory infiltrates, 3+confluent inflammation and 4+diffuse inflammation spread throughout the cut). The values of two cuts were summed to obtain an average inflammatory value.

Subsequently the development of fibrosis was assessed using Masson's trichrome staining. For this purpose, the presence and the distribution pattern of collagen fibers was determined, awarding a value of 1+to the increase of interstitial fibrous tissue surrounding bundles of muscle fibers and a value of 2+to the presence of fibrous tissue surrounding and isolating atrophic individual muscle fibers or patches of dense fibrous tissue occupying a space suggesting that it has corresponded to a missing muscle fiber. The results are summarized in Tables 1 and 2.

TABLE 1

| | Heart | | | | | Muscle | | |
|---|---|---|---|---|---|---|---|---|
| Mouse | | Inflammation | Fibrosis | Total | PCR | Inflammation | Calcium | PCR |
| Control | | | | | | | | |
| 123-1 | | | | | 0.043 | 2 | 0 | 3.01 |
| | VI | 2 | 1 | 3 | | | | |
| | T | 2 | 2 | 4 | | | | |
| | Average | 2 | 1.5 | 3.5 | | | | |
| 123-3 | | | | | neg | 2 | 2 | neg |
| | VI | 2 | 2 | 4 | | | | |
| | T | 2 | 2 | 4 | | | | |
| | Average | 2 | 2 | 4 | | | | |
| TS mut | | | | | | | | |
| 125-1 | | | | | neg | 3 | 0 | 8.86 |
| | VI | 2 | 2 | 4 | | | | |
| | T | 0 | 0 | 0 | | | | |
| | Average | 1 | 1 | 2 | | | | |
| 125-4 | | | | | neg | 0 | 1 | 0.064 |
| | VI | 2 | 2 | 4 | | | | |
| | T | 0 | 1 | 1 | | | | |
| | Average | 1 | 1.5 | 2.5 | | | | |
| 125-5 | | | | | neg | 2 | 0 | 0.53 |
| | VI | 1 | 2 | 3 | | | | |
| | T | 1 | 2 | 3 | | | | |
| | Average | 1 | 2 | 3 | | | | |
| 126-1 | | | | | neg | 2 | 0 | neg |
| | VI | 2 | 1 | 3 | | | | |
| | T | 2 | 2 | 4 | | | | |
| | Average | 2 | 1.5 | 3.5 | | | | |
| 126-2 | | | | | neg | 1 | 0 | neg |
| | VI | 2 | 2 | 4 | | | | |
| | T | 2 | 1 | 3 | | | | |
| | Average | 2 | 1.5 | 3.5 | | | | |
| 126-3 | | | | | neg | 0 | 1 | ND |
| | VI | 2 | 2 | 4 | | | | |
| | T | 1 | 1 | 2 | | | | |
| | Average | 1.5 | 1.5 | 3 | | | | |
| 126-4 | | | | | 0.035 | 0 | 2 | ND |
| | VI | 2 | 2 | 4 | | | | |
| | T | 2 | 2 | 4 | | | | |
| | Average | 2 | 2 | 4 | | | | |
| 127-11 | | | | | neg | 0 | 1 | ND |
| | VI | 2 | 1 | 3 | | | | |
| | T | 1 | 0 | 1 | | | | |
| | Average | 1.5 | 0.5 | 2 | | | | |
| 127-12 | | | | | neg | 1 | 0 | neg |
| | VI | 3 | 2 | 5 | | | | |
| | T | 2 | 0 | 2 | | | | |
| | Average | 2.5 | 1 | 3.5 | | | | |
| TS E Coli | | | | | | | | |
| 130-3 | | | | | neg | 2 | 0 | neg |
| | VI | 2 | 1 | 3 | | | | |
| | T | 2 | 1 | 3 | | | | |
| | Average | 2 | 1 | 3 | | | | |
| 130-6 | | | | | neg | 2 | 0 | ND |
| | VI | 2 | 1 | 3 | | | | |
| | T | 2 | 1 | 2 | | | | |
| | Average | 2 | 1 | 2.5 | | | | |
| 131-7 | | | | | neg | 2 | 0 | neg |
| | VI | 2 | 0 | 2 | | | | |
| | T | 2 | 2 | 4 | | | | |
| | Average | 2 | 1 | 3 | | | | |
| TS WT | | | | | | | | |
| 128-1 | | | | | 0.114 | 3 | 0 | 0.103 |
| | VI | 2 | 2 | 4 | | | | |
| | T | 2 | 2 | 4 | | | | |
| | Average | 2 | 2 | 4 | | | | |
| 128-2 | | | | | 0.056 | 3 | 1 | 24.03 |
| | VI | 2 | 1 | 3 | | | | |
| | T | 1 | 1 | 2 | | | | |
| | Average | 1.5 | 1 | 2.5 | | | | |
| 128-4 | | | | | neg | 1 | 0 | neg |
| | VI | 2 | 0 | 2 | | | | |
| | T | 2 | 1 | 3 | | | | |
| | Average | 2 | 0.5 | 2.5 | | | | |
| 129-9 | | | | | neg | 0 | 1 | 0.063 |
| | VI | 2 | 1 | 3 | | | | |
| | T | 2 | 1 | 3 | | | | |
| | Average | 2 | 1 | 3 | | | | |
| 127 | | | | | neg | 2 | 0 | 2.31 |
| | VI | 1 | 1 | 1 | | | | |
| | T | 1 | 1 | 1 | | | | |
| | Average | 1 | 1 | 1 | | | | |

PCR: parasitic equivalents/ng;
VI: left ventricle;
T: Septum

Spleen histopathologic evaluation was performed based on the distribution, size and morphology of the white pulp and characteristics of the population of cells in red pulp. Morphological findings in the spleen are indicative of an immune activation state.

TABLE 2

| Mouse | Heart | Skeletal muscle | Spleen |
|---|---|---|---|
| 123-1 | Unspecified chronic | Unspecified active chronic | Reactive unspecified |
| | myocarditis | myocarditis | follicular lymphoid |
| | | | hyperplasia |
| 123-3 | Unspecified chronic | Unspecified active chronic | Reactive unspecified |
| | myocarditis | myocarditis | follicular lymphoid |
| | | | hyperplasia |
| 125-1 | Unspecified chronic | Unspecified active chronic | Reactive unspecified |
| | myocarditis | myocarditis | follicular lymphoid |
| | | | hyperplasia |
| 125-4 | Unspecified active | Non-significant | Reactive unspecified |
| | chronic myocarditis | histopathological changes | follicular lymphoid |
| | | of musculoskeletal system | hyperplasia |
| 125-5 | Unspecified chronic | Unspecified active chronic | Reactive unspecified |
| | myocarditis | myocarditis | follicular lymphoid |
| | | | hyperplasia |
| 126-1 | Unspecified chronic | Unspecified active chronic | Reactive unspecified |
| | myocarditis | myocarditis | follicular lymphoid |
| | | | hyperplasia |
| 126-2 | Unspecified chronic | Unspecified active chronic | Reactive unspecified |
| | myocarditis | myocarditis | follicular lymphoid |
| | | | hyperplasia |
| 126-3 | Unspecified chronic | Non-significant | Reactive unspecified |
| | myocarditis | histopathological changes | follicular lymphoid |
| | | of musculoskeletal system | hyperplasia |
| 126-4 | Unspecified chronic | Skeletal Muscle with | Reactive unspecified |
| | myocarditis | multifocal calcifications | follicular lymphoid |
| | | of muscular fibers | hyperplasia |
| 127 | Unspecified chronic | Unspecified active chronic | Reactive unspecified |
| | myocarditis | myocarditis | follicular lymphoid |
| | | | hyperplasia |
| 127-11 | Unspecified chronic | Non-significant | |
| | myocarditis | histopathological changes | |
| | | of musculoskeletal system | |
| 127-12 | Unspecified chronic | Unspecified active chronic | Reactive unspecified |
| | myocarditis | miocarditis | follicular lymphoid |
| | | | hyperplasia |
| 128-1 | Unspecified chronic | Unspecified active chronic | Reactive unspecified |
| | myocarditis | myocarditis | follicular lymphoid |
| | | | hyperplasia |
| 128-2 | Unspecified chronic | Unspecified active chronic | Reactive unspecified |
| | myocarditis | myocarditis | follicular lymphoid |
| | | | hyperplasia |
| 128-4 | Unspecified chronic | Unspecified active chronic | Reactive unspecified |
| | myocarditis | myocarditis | follicular lymphoid |
| | | | hyperplasia |
| 129-9 | Unspecified chronic | Non-significant | Reactive unspecified |
| | myocarditis | histopathological changes | follicular lymphoid |
| | | of musculoskeletal system | hyperplasia |
| 130-3 | Unspecified chronic | Unspecified active chronic | Reactive unspecified |
| | myocarditis | myositis | follicular lymphoid |
| | | | hyperplasia |
| 130-6 | Unspecified chronic | Non-significant | Reactive unspecified |
| | myocarditis | histopathological changes | follicular lymphoid |
| | | of musculoskeletal system | hyperplasia |
| 131-7 | Unspecified chronic | Unspecified active chronic | Reactive unspecified |
| | myocarditis | myositis | follicular lymphoid |
| | | | hyperplasia |
| Control | Myocardial tissue | Non-significant | Splenic parenchyma |
| N-1 | without | histopathological changes | without significant |
| | histopathological | of musculoskeletal system | histopathological |
| | alterations signs | | alterations |
| Control | Myocardial tissue | Non-significant | Splenic parenchyma |
| N-2 | without | histopathological changes | without significant |
| | histopathological | of musculoskeletal system | histopathological |
| | alterations signs | | alterations |

Tissue Parasitic Charge Determined by PCR in Real Time

It was conducted with approximately 50 mg of tissue (heart and skeletal muscle). The sample was kept in DNAzol (Invitrogen) and was processed by mechanical disintegration by TissueRuptor. DNA precipitated with isopropanol, was washed with 70% ethanol and resuspended in NaOH and HEPES, according to the manufacturer's specifications.

Quantification by Real-Time PCR

The protocol published by Cummings and Tarleton in “Molecular and Biochemical Parasitology” 129 (2003) 53-59 was followed.

### BRIEF DESCRIPTION

Detection and quantification of parasites in the sample were established by amplification of parasite DNA, using satellite region (SAT) of the T. cruzi genome as “target” for the reaction. Oligonucleotides TCZ-F (GCTCTTGCCCACAMGGGTGC) (SEQID NO:2) and TCZ-R (CCAAGCAGCGGATAGTTCAGG) (SEQID NO:3) were used, which amplify a fragment of 182 pb. This region is in thousands of copies per genome, which increases the detection sensitivity.

For quantification of genomic DNA of mice, a fragment of the TNFα gene (oligonucleotides TNF-5241 (5-TCCCTCTCATCAGTTCTATGGCCCA-3) (SEQID NO:4) and TN F-5411 (5-CAGCAAGCATCTATGCACTTAGACCCC-3) (SEQID NO:5) was amplified. This is a single copy gene, which allows its use as normalizer of the loading and amplification process during the reaction.

Calibration curves for both sequences were performed. The TNF curve was performed using mixtures of DNA from the samples to be analyzed (and subsequent serial dilutions). This allowed quantitation in the range of 200-0.02 ng DNA. The SAT curve was performed using DNA from healthy tissue (uninfected animals), contaminated with known amounts of parasites (and subsequent serial dilutions). This allowed quantitation in the range of 400 to 0.04 parasitic equivalents. Quantification was expressed in parasite equivalents/DNA mass. To this effect, it was normalized to long and 50 ng for skeletal muscle and heart, respectively.

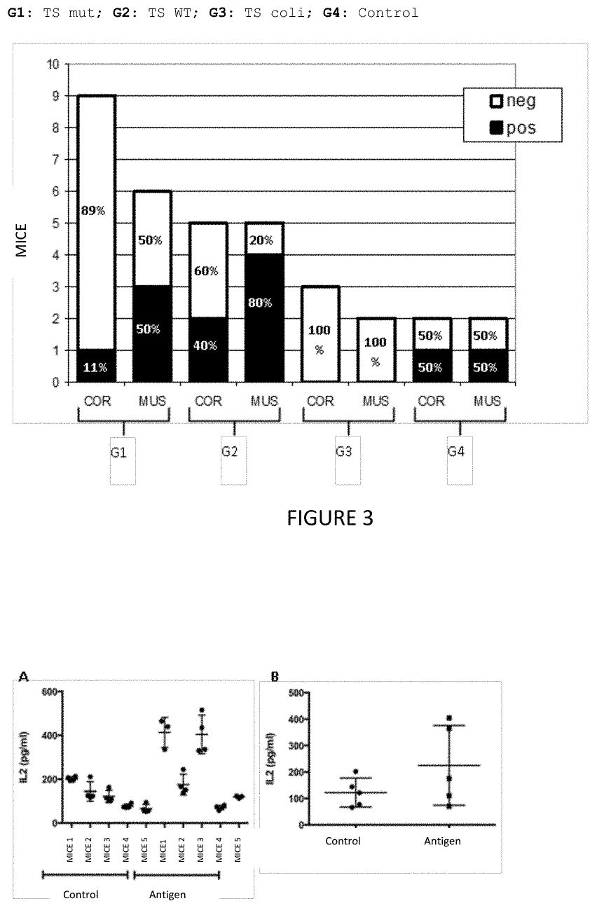

The results (number of parasite equivalents/ng DNA) obtained by RT PCR in four groups of study are presented in FIG. 3.

Test 2

Cytokine Profile Determination in Animals Immunized with TSmut (SEQID NO:1)

Male, 60 days of age BALB/cj mice were used. Five (5) animals received 15 ug of TSmut (SEQID NO:1) per mouse via s.c., diluted in PBS and emulsified 1:1 with adjuvant ISA51 (Seppic, France) (100 ul emulsion/mouse). Another group of five (5) animals received only PBS emulsified in the same adjuvant. Mice were sacrificed at day +5 post-immunization. Splenocyte cultures were performed (5×10 ⁶ cells/ml) in RPMI 1640 supplemented with 10% fetal bovine serum at 37° C. and 5% CO₂. The cultures were stimulated for 72 hours with the same antigen (5 ug/ml) and supernatants were harvested 24 hours later. For each mouse cultures were performed in quadruplicate. The concentration of IL2, IL4 and IFNg in the culture supernatants was measured by sandwich ELISA with monoclonal antibody pairs for capture and detection Biolegend (CA, USA).

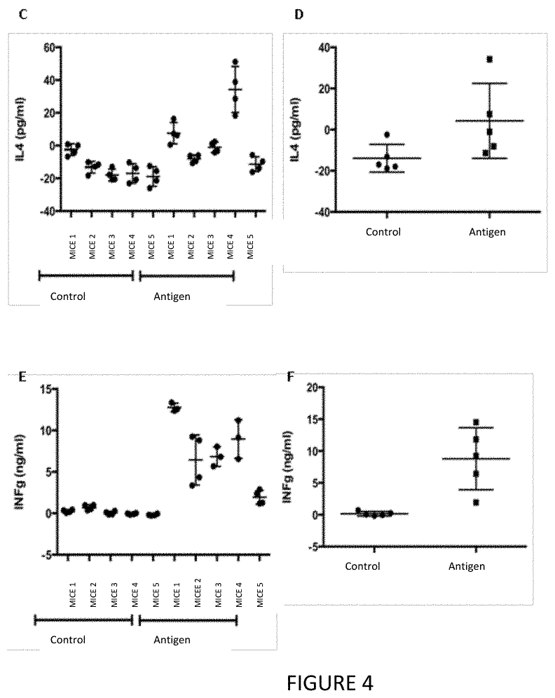

In FIG. 4 the results of quantitation of IL2, IL4 and IFNg were observed by ELISA. (A, C, E) Each point represents quadruplicate values for each supernatant. (B, D, F) Each point represents the average of quadruplicate. Cultures were harvested 24 hours after adding 5 ug/ml of TSmut. Control: Animals immunized with PBS/ISA51. TSmut (SEQID NO:1) Animals immunized with TSmut/ISA51.

By ex vivo restimulation it was possible to characterize the specific response to antigen as a clear response Th1 by significant production of IFNg of treated animals compared to control animals (FIGS. 4E and 4F).

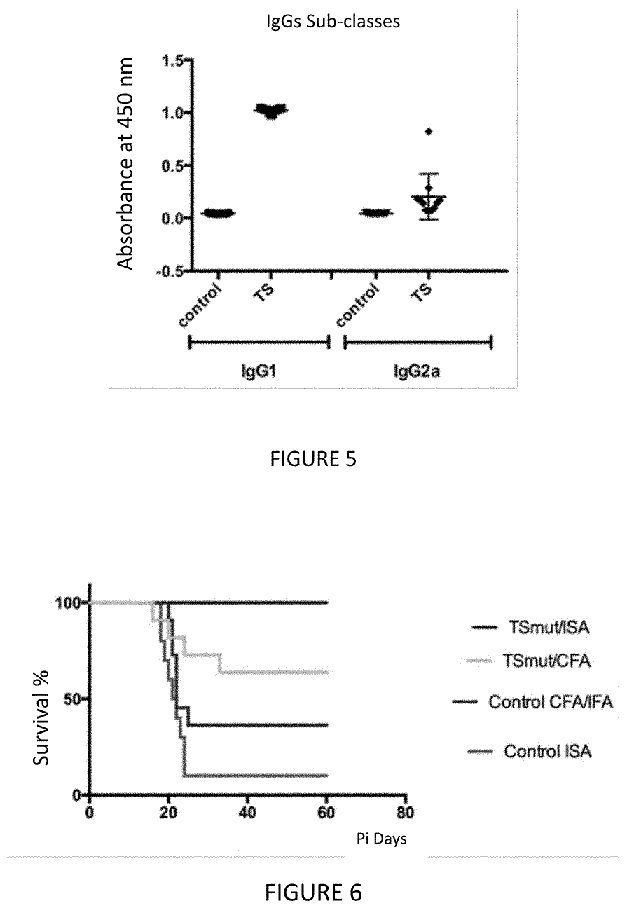

There is strong evidence supporting the importance of Th1 response as crucial to the survival of T. cruzi infection. However, when this cell response is triggered, it is very detrimental to the host, due to damage that can result in tissue. Currently, there is an amount of evidence that indicate that the actual protective response should add balanced phenotypes of Th1/Th2 CD4 T cells to restrict the spread of parasites, but avoiding substantial damage to infected tissue (Ruiz Diaz, 2015). (FIG. 5)

Test 3

An assay to compare Freund's adjuvant and Montanide® ISA 51VG (Seppic, France), an adjuvant approved for human and tested in Test 2 was carried out. It was assessed by parasitemia and mortality.

Mice 60 days of age received three doses of 15 ug of TS-mut (SEQID NO:1) administered s.c. with two week intervals. Freund's adjuvant (CFA/IFA) was emulsified in CFA (1:1 vol:vol) in the first dose and IFA (1:1, vol:vol) the other two. Montanide® ISA 51VG emulsified 1:1 with the antigen.

Group A
n = 11
Days

| 1 | 15 | 30 | 45 | 105 |
|---|---|---|---|---|
| MUT/CFA | MUT/IFA | MUT/IFA | Challenge | Parasitemia/Mortality |

Group B
n = 11
Days

| 1 | 15 | 30 | 45 | 105 |
|---|---|---|---|---|
| CFA | IFA | IFA | Challenge | Parasitemia/Mortality |

Group C
n = 10
Days

| 1 | 15 | 30 | 45 | 105 |
|---|---|---|---|---|
| MUT/ISA | MUT/ISA | MUT/ISA | Challenge | Parasitemia/Mortality |

Group D
n = 10
Days

| 1 | 15 | 30 | 45 | 105 |
|---|---|---|---|---|
| ISA | ISA | ISA | Challenge | Parasitemia/Mortality |

Animals were challenged a day 45 after the first immunization with a virulent strain T. cruzi DTU TCVI (RA), by administering blood trypomastigotes 500 via i.p. Parasitemia and mortality were monitored for 60 days. Parasitemia was determined by counting the parasites in a hemocytometer (Neubauer). Values are expressed as parasites/ml.

The results obtained in terms of survival and parasitemia are shown in FIG. 6 and Table 3, respectively:

TABLE 3
Parasitemia Values

| | TSmut | Control | | |
|---|---|---|---|---|
| Group | CFA/IFA | CFA/IFA | TSmut ISA | Control ISA |
| Mouse n^{o} | (×10⁶) | (×10⁶) | (×10⁶) | (×10⁶) |
| 1 | 0.5 | 0.2 | 0.65 | 4.1 |
| 2 | 0.5 | 0.2 | 0.5 | 4.9 |
| 3 | 0.4 | 0.3 | 0.35 | 3.4 |
| 4 | 1 | 0.2 | 0.45 | 6.9 |
| 5 | 1.5 | 1.4 | 0.4 | 0.5 |
| 6 | 0.35 | 1.85 | 0.3 | 1.1 |
| 7 | ND | 2.9 | 0.1 | 0.55 |
| 8 | 0.5 | 1.25 | 0.35 | 2 |
| 9 | 7.5 | 0.35 | 0.3 | 2.15 |
| 10 | 0.3 | 1.25 | 0.1 | 8.5 |
| 11 | 0.4 | 2.85 | | |

Parasitemia values are expressed as number of parasites/ml and correspond to the 17 pi (RI to R5) and 20 pi (R6 RLL) days.

The results were obtained using male BALB/c mouse model and Fontanella et al. (2008). Three doses of immunogen were used. In our tests, mice were infected with the RA strain of parasite (TcVI) instead of the Tulahuen strain. Survivals with TSmut were consistent in both studies. In our assays, histological analysis and the parasite load animals were performed after re-challenging animals with a higher number of parasites, therefore they are not comparable with those reported previously.

Referring to article Bontempi et al. (2015), the authors used female BALB/c mice and reduced doses of antigens to 10 ug each.

We also found a predominant Th1 immune response, which was raised immediately after a single dose of immunogen.

When a comparison test was performed between adjuvants, the TSmut significantly reduced parasitemia vs. control adjuvant Montanide® ISA 51. Replacing CFA by ISA 51 offered a better overall protection in terms of parasitemia and survival. A 100% survival was recorded in the TSmut/ISA group vs 63-82% in TSmut CFA/IFA immunization test.

1. A vaccine composition against infection by Trypanosoma cruzi comprising: at least one Trypanosoma cruzi trans-sialidase mutant protein and, as adjuvant, a mixture of a purified mineral oil and mannide monooleate.

2. A vaccine composition against infection by Trypanosoma cruzi according to claim 1, wherein said Trypanosoma cruzi trans-sialidase mutant protein has a sequence SEQID NO:1.

3. A vaccine composition against infection by Trypanosoma cruzi according to claim 1, wherein said purified mineral oil is the one marketed as Drakeol 6VR.

4. A vaccine composition against infection by Trypanosoma cruzi according to claim 1, wherein said Trypanosoma cruzi trans-sialidase mutant protein has a sequence SEQID NO:1 and wherein said mixture of a highly purified mineral oil and mannide monooleate is the one marketed as Montanide ° ISA 51 VG.
